(19) United States
(12) Patent Application Publication
(10) Pub. No.: US 2002/0072734 A1
(43) Pub. Date: Jun. 13, 2002

(54) Chip systems for the controlled emission of substances having a chemosensory effect

(75) Inventor: Rainer K. Liedtke, Munich (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: PHARMED HOLDING GMBH, Munich (DE)

(21) Appl. No.: 10/006,666

(22) Filed: Dec. 10, 2001

(30) Foreign Application Priority Data
Dec. 8, 2000 (DE) 100 61 057.9

(51) Int. Cl. A61K 9/22

(52) U.S. Cl. 604/890.1

(57) ABSTRACT

A biological chemosensorial reaction can be produced using a process and a chip system for the controlled emission of a substance or a mixture of substances that work chemosensorially. The chip system emits, in a controlled as well as programmable manner, the substance or a mixture of substances that trigger a biochemical transduction at a chemosensory receptor for a neuronally transmitted signal to a specific structure of the central nervous system. The new process and its devices render numerous new applications possible within the medical and biological areas for chemosensorially active substances.

### BACKGROUND OF THE INVENTION

1. Field of the Invention

The invention relates to a process and a chip system for the controlled emission of a substance or a mixture of substances having a chemosensory effect.

2. Discussion of the Background

In the field of biology it is known that many plants emit natural substances as signals that produce biological reactions in insects which have biosensors for these signals, such as chemosensory antenna organs. Likewise, insects can emit chemical signaling substances into the environment. They can thereby communicate, for example, regarding the search for food or for the purpose of reproduction.

Similarly, in the area of human beings and in the realm of mammals it is known that considerable influences upon bodily functions can be produced by chemosensory means with the aid of substances that cause olfactory activity, particularly in the area of emotions. The first biological starting point for such chemosensory active stimuli is the so-called regio olfactoria of the apparatus of the sense of smell. This regio olfactoria is an anatomical region that lies in the upper posterior region of the third turbinal concha, in the nasal cavity. Its surface, which is turned toward the airway of the nasal cavity, consists of primary sensory cells which represent a field of chemo-receptors at which a so-called transduction occurs, a biochemical transformation of the receptor reaction into the triggering of electrical power. The central nervous system's processing center for the stimulus signals from there, which undergo intermediate processing by way of the reversing station of the bulbus olfactorius, is the limbic system. The latter plays a coordinative as well as an integrating role of an “emotional brain,” particularly in the interplay with a few other structures, especially the hypothalamus and the cortical projections.

Within the context of various biological and medical investigations involving mostly natural scent-bearing substances that are inhaled trans-nasally, it was possible to demonstrate both mood changes and changes in the EEG (Diego MA et al., Int. J. Neurosci. 1998 96 (3/4) 217). The changes in the EEG also identified relationships to the nature of the smells (Lee, CF et al., Ann. Physiol. Anthrop. 1994 13 (5) 281; Harada, H. et al., Clin. Electroencephalogr. 1998 29 (2) 96). It was even possible to ascertain neurophysiological effects in the olfactory brain of the rat (Zibrowski E. M. et al., Brain Res. 1998 800 (2) 207). It should be pointed out that eating behavior and reproduction are addressed more specifically and likewise only by way of example. Thus, it was ascertained that overweight women react to exposure to the smell of food with a different pattern of salivation than was the case among women of normal weight (Epstein L H et al., Psychosom. Med. 1996, 58 (2) 160). Among primates, it was possible to demonstrate interactions of the orbitofrontal cortex with electro physiological reactions of olfactory neurons to food smells (Critchley H D et al., J. Neurophysiol., 1996 (75(4) 1673); O'Doherty J. et al., Neuroreport 2000, 11(2) 399). It was also shown, that among other things, olfactory stimuli appear as signals to nourishment, that these occurred even prior to the intake of nourishment (Bray G A, Proc. Nutr. Soc., 2000, 59(3) 373). Further, female mice that have copulated and thereafter were exposed to the scent of a strange male are subject to such hormonal changes as a result of which a pregnancy block occurs (Kaba, H., et al., Neuroscience 1988, 25(3) 1007; Li, C S. et al., Neurosci. Letter 1994, 176 (1) 5; Kaba H. et al., Science 1994, 265 (5169) 262).

The fact that central effects are not limited to substances that are generally regarded as “scents” in pharmacology seems to apply to many other substances as well. For example, surprisingly the neuropeptide oxytocin, which is regarded primarily as a type of hormone that is specific to the uterus and the mammary gland, triggered rapid effects in the central nervous system that relieved anxiety. These effects were anti-depressive and anti-aggressive in mice and rats, when oxytocin was applied to the regio olfactoria. The triggering of afferent nerve signals to certain involved hypothalamus nuclei was demonstrated as well (L D. Neuman et al., Abstract XXXIst Congress of the Internal. Society of Psychoneuroendocrinology (ISPNE) Melbourne, October 2000).

It thus becomes apparent from these examples that far-reaching pharmacodynamic effects can be triggered via an olfactory route.

However, in order to be able to use substances that work in a chemosensory manner in a way that is suitable for triggering bio-reactions and to cause biological effects, especially for therapeutic or diagnostic applications, it is necessary that the substances also be emitted in a form that is suitably controlled. This cannot be achieved, however, with the devices that have been known thus far.

Access to the sensory regio olfactoria is determined by the anatomical conditions of this specific region, but especially by the fact that there are chemo-receptors on nervous structures. This also explains the high degree of sensitivity of this region, as a result of which only a small dose is required to trigger effects. The access to the sensory regio olfactoria is furthermore determined by the fact that it is an anatomically small and concealed localized surface, which can be reached only by air pathways. In accordance with the biologically natural functions of this region, a form of application by which a substance can reach this region requires a suitably volatile form. Thus, formulations having the most finely dispersed particles floating in gases are required. This allows the chemo-sensorially active substances to be transported to this specific receptor area.

One technical possibility are formulations that are customarily referred to as nasal sprays. However, among the conventional nasal sprays currently available, the preponderant portion of the active ingredients contained within these formulations reaches only the absorptive area of tile mucous membrane in the region of the lower nasal cavity. In this instance, this is intended from a pharmaceutical and technical viewpoint. The pharmacokinetic function of nasal sprays, as one of the variants of systemic administration, is to promote the transport of the substance into the blood by way of the non-specific nasal mucous membrane. In another instance, the pharmacokinetic function of nasal sprays is to cause a local deposition of the substances in order to engender topical effects. By contrast, the regio olfactoria lies higher topographically. It possesses as a chemosensorial nervous organ and no absorptive epithelium, but has actually a secretory function.

So-called smelling strips are commonly used in perfumeries, or by perfume developers for smelling tests. In that case, certain scent-bearing substances are dropped onto absorbent paper strips, and these are then held to the nose for the purpose of a crude qualitative trial of the smell. However, aids of this type allow neither an exact dosage of the substance, nor a controlled evaporation that is reproducible. Among other things, they exhibit immediate losses due to uncontrolled evaporation, and they cannot be stored. Further, it is well known that UV light destroys many, if not most, scent-bearing substances. Thus, methods such as the simple, unprotected application of substances onto papers or films are unsuitable. An improved form of such smelling papers has slightly greater storage capabilities. These improved smelling papers are also used as scent samples, in which the scent-bearing substances are first filled into small, capsule-like particles prior to being applied to the paper. The shell of the particles then tears when external mechanical pressure is applied. Following this, however, the scent-hearing components enclosed therein escape completely and in an uncontrolled manner. In addition, they are exposed to thermal influences and to the effects of light.

Therefore, none of these attempts thus far has satisfied the technical requirements that must be imposed upon a controlled system or the requirements of therapeutic, diagnostic, or biologically controlled applications.

### SUMMARY OF THE INVENTION

It is therefore an object of the present invention to achieve a controlled emission of substances that have a chemosensorial effect, especially for the production of biological chemosensory reactions.

This and other objects of the present invention have been achieved by the first embodiment of the present invention which includes a chip system for the controlled emission of a substance having a chemo sensory effect, comprising:

a chip carrier matrix;

a carrier layer comprising the substance having a chemosensory effect;

a promoter layer comprising an emission promoter; and

an emission control layer, which is a membrane or a polymer matrix.

In another embodiment the present invention relates to a process for the controlled emission of a substance having a chemo sensory effect, comprising:

releasing said substance or a mixture of at least two substances having a chemosensory effect from the chip system according to claim 1 as a volatile substance or a volatile mixture of at least two substances;

wherein a thermodynamic diffusion activity of said substance or said mixture of at least two substances is controlled by said promoter; and

wherein a rate of emission of said substance or said mixture of at least two substances is controlled by a layer that serves as a diffusion control;

wherein a promotion of said emission is governed chemically, physically, or biologically.

### BRIEF DESCRIPTION OF DRAWINGS

FIG. 1 shows a schematic cross-section of the basic structure of a chip system.

FIG. 2 shows a schematic cross-section of the structure of a chip system.

FIG. 3 shows the exploded schematic representation of a chip system.

FIG. 4 shows a schematic cross-section of the structure of a chip system.

FIG. 5 shows a schematic c ross-section of the structure of a chip system.

FIG. 6 shows a parallel application of operational layers in a chip system.

FIG. 7 shows the exploded schematic representation of a chip system.

### DETAILED DESCRIPTION OF THE INVENTION

Controlled emission of substances that have a chemosensorial effect can be achieved by using a chip system for the purpose of controlled emission of a chemosensory substance. This chip system is a multiple component system which is comprised in such a way that the chemosensory active substance or a mixture of at least two substances is released in a controlled manner, for example, as a volatile mixture of at least two substances. The thermodynamic diffusive activity of the substances is controlled by an adjuvant that serves as promoter. The substance's rate of emission is controlled by a layer that serves as a diffusion control. The promotion can be controlled chemically, physically, or biologically. A corresponding device for the controlled emission of a substance or a mixture of substances is comprised of a carrier layer with the chemosensorially active substances and a layer that contains the promoter. This layer can be connected with additional layers that contain devices that assist promotion. The device further comprises an emission control layer which may be a membrane or a polymer matrix, as well as a carrier matrix, housing or casing comprising all the components. On the external underside of this carrier matrix an adhesive layer may be found for the purpose of affixing the device. A covering layer which is removable from the emission control layer may be found on the top of this carrier.

In a second embodiment of the invention, the control of the adjuvants, which serve as promoters occurs mechanically, thermally, electrically, magnetically, biologically, chemically, or bio-chemically, or as a combination of such measures, in order to improve and expand practical use. This control preferably occurs in such a way that the regulation of the promotion can occur in various series of programs. The regulation may be based on open loop or closed loop technology.

Solutions of ethanol, distilled oils, chemical synthetic substances, technical or natural gases are used as promoters in a third embodiment of the invention. The chemical synthetic substances preferably possess physical chemical properties comparable to ethanolic or distilled oils. The promoters are initially preferably present in a different aggregate state. They are preferably used individually or in the form of combinations.

In a fourth embodiment of the present invention the membranes or matrices that are used for emission control consist of natural substances or synthetic polymers, such that the latter may have openings for emission. The openings are applied by mechanical aids, or by laser techniques and the diameter of the openings can be regulated separately and dynamically.

In a fifth embodiment of the invention, the functional layers of the chemosensorially active ingredient and the promoter are housed in a commonly shared matrix layer.

In a sixth embodiment of the invention, the spatial dimensions of the chip system preferably lie within the range of centimeters to micrometers. Preferably, the chip system has dimensions of from 0.001 micrometer to 100 centimeter, more preferably from 1 micrometer to 10 centimeter and even more preferably from 10 micrometer to 1 centimeter. The dimensions include all values and subvalues therebetween, especially including 0.05, 0.1, 0.5, 1, 5, 10, 50, 100 micrometer and 1, 5, 10, 50 and 100 centimeter.

In a seventh embodiment of the invention, the chip system is combined directly with a technical device that influences or controls the quantity and composition of the air, especially with a device to affect air flow, air temperature, ionization of the air, air filtration, aromatization of the air, air humidity, and the admixture of air with liquids, solids, or gases.

In an eighth embodiment of the invention, flexible, expandable, transparent, electrically conductive, light emitting or light absorbing, heat-storing or heat-emitting substances; magnetic materials, electrically conductive metal foils, electrically conductive plastics, electronic components, porous natural or synthetic polymers may be processed as materials for components.

In a ninth embodiment of the invention the chip system has a device with which the present content or the biological activity of the chemosensorially active substances or their products associated with them, or the products of their degradation, is determined or displayed.

In a tenth embodiment of the invention the chip systems are expanded to complex combinations of several identical or varied chip systems.

In an eleventh embodiment of the invention preferably all natural and chemically synthetic chemosensorially active substances are used which are well-suited for influencing disorders of the central or vegetative nervous system, especially behavioral disorders, symptoms of stress, sleep disorders, disorders involving anxiety and aggression, eating and weight-related disorders, sexual disorders, reproductive disorders, pain, vascular and circulatory disorders occurs. The natural or chemically synthetic chemosensorially active substances may be present as individual substances, or in combinations with one another.

In a twelfth embodiment of the invention preferably all substances are used which have an effect in the realm of the human, the animal or plants and the vegetable, as well as all environmental systems in biological regulatory processes that work as chemosensorially active signaling substances, including their natural or synthetic analogs, metabolites, derivatives, isomers, and antagonists. The substances may be used singly or in combination.

In a thirteenth embodiment of the invention chemosensorially active substances are used which are contained within pharmaceutical products and products for veterinary medicine, in medical aids, products for health care or care of the body, cosmetic articles or perfume articles, natural or artificial foodstuffs and beverages, dietetic products, plants and agricultural products, biological, microbiological, and chemical products for agricultural, horticultural, forest management, and marine management purposes, agents to fend off or eliminate harmful animals, plants or micro-organisms, household articles, sporting articles, and articles for use, packaging materials, articles of clothing, including all their natural or synthetic chemical analogs, metabolites, derivatives, isomers, or antagonists. They may be used singly or in combination.

Advantages of the invention are due to the fact that the described chip systems can liberate the chemosensorially active substances in reproducible fashion in prescribed doses even over longer periods of time. In addition, the emission system may be passive or active. In the case of passive systems, the conditions of emission are prescribed by virtue of the corresponding parameters of the components, for example, the porosity of the membrane, the nature of the promoters, aggregate circumstances, etc. Active chip systems are possible based on internal implementation of guidance and control components. Thus, the process of controlled emission can also occur in a programmed fashion. Various control devices are housed within a chip system, both for the emission itself as well as for the processes for its promotion. Controls over thermal processes in particular, controlled influences of thermodynamic activities, play a crucial role. Thus, for example, minimal levels of chemosensorial substances that cannot be detected physiologically often become physiologically detectable in the heated transport phases, that is, they reach a chemosensorially active potential. Depending upon the requirement and the goal of application, the control elements of the chip systems can be implemented either in all, or only in constituent processes, and be embodied either as “open loop” systems or as back coupling “closed loop” systems. Different applications are rendered possible and liberation kinetics can also be exactly adapted to the neurophysiological conditions of organs that are chemosensorially receptive. For example, emissions can pulse at predetermined intervals of time. In this way, adaptive counter-reactions can be decreased. Similarly, various measurement systems for monitoring activity can also be integrated into the chip systems, for example, for the display of the substances and their current content. Statements regarding current biological activity result may result from such measurements.

Individual chip systems can be combined to complex chip groups in expanding modular fashion. The use of numerous additional combinations of substances is possible. Thus, as a result of such complex combinations, emission patterns can be both qualitatively modified or quantitatively enhanced. Furthermore, the chip systems can also be combined with external devices, for example, with external heat sources, or with such devices that influence the direction and amplitude of convection of the substances that are emitted, for example, at ventilation facilities. Furthermore, the chip systems can also be used as technical components in conventional dosing devices, or in other technical devices, such as in measured dose spray pumps, for example.

The chip systems render applications possible in various biological investigations: within the context of applications providing therapy for human beings, for example for mental mood disorders. The triggering of biologically chemosensorial reactions exhibits significant advantages over the effects of a systemic administration of the customary psycho-pharmaceuticals. Psycho-pharmaceuticals that act upon the central nervous system produce a considerable number of adverse side effects, including various forms of diminished performance. In addition, their therapeutic effects are often not discernible until weeks after beginning of the systemic administration. For example, an oral application with tablets entails the distribution of the ingested substance throughout the entire body. Only a small portion of the substance that was administered, ultimately reaches its target in the central nervous system to the extent that it can overcome the blood-brain barrier. Consequently, such effects are very unspecific and they cannot be readily controlled. By contrast, the chemosensory chip systems cause the chemical molecular information of the substances that are emitted to be transformed, quickly and selectively, into neuronal activities by means of a direct and biologically acting mechanism by way of the olfactory route. In addition, this neuronal process produces, with far lower doses, a more rapid occurrence of the effect, thus a higher degree of certainty of treatment. Chip systems made of and/or with flexible or expandable materials or components can be affixed to the body, in a reversible way, just like an adhesive bandage.

As a further example of the breadth of application, the chip systems can also be applied to objects that are near the body or clothing for the purpose of emitting chemosensorially acting repellents against sticking or sucking insects. Thus, the danger of the transmission of infections, such as malaria, can be diminished. Additional possibilities of biological use lie in the realm of environmentally friendly protection against organisms that are harmful to plants.

Another technical advantage is that the chip systems can be produced economically in rather large quantities by the usual means of production and in accordance with exact norms in a reproducible manner. Furthermore, depending upon the application, they can be given various dimensions, even down to micro-scale. Forms of production such as those that are used in printing techniques or in the area of microelectronics may be used for mass production. Production of small series, as well as production in accordance with individual instructions is possible by these means. Overall, they also lend themselves well to such technical requirements as the mandated standards for a production of pharmaceutical or biotechnical products demand.

As a result of their embodiment possibilities, the chip systems furthermore afford controlled storage conditions and stability and hygiene even for sensitive substances, such as photo-sensitive, temperature-sensitive and oxidation-sensitive substances. Furthermore, they can be adapted to varied environmental conditions, for example, they may be impervious to dust or watertight.

Having generally described this invention, a further understanding can be obtained by reference to certain specific examples which are provided herein for purposes of illustration only, and are not intended to be limiting unless otherwise specified.

### EXAMPLE

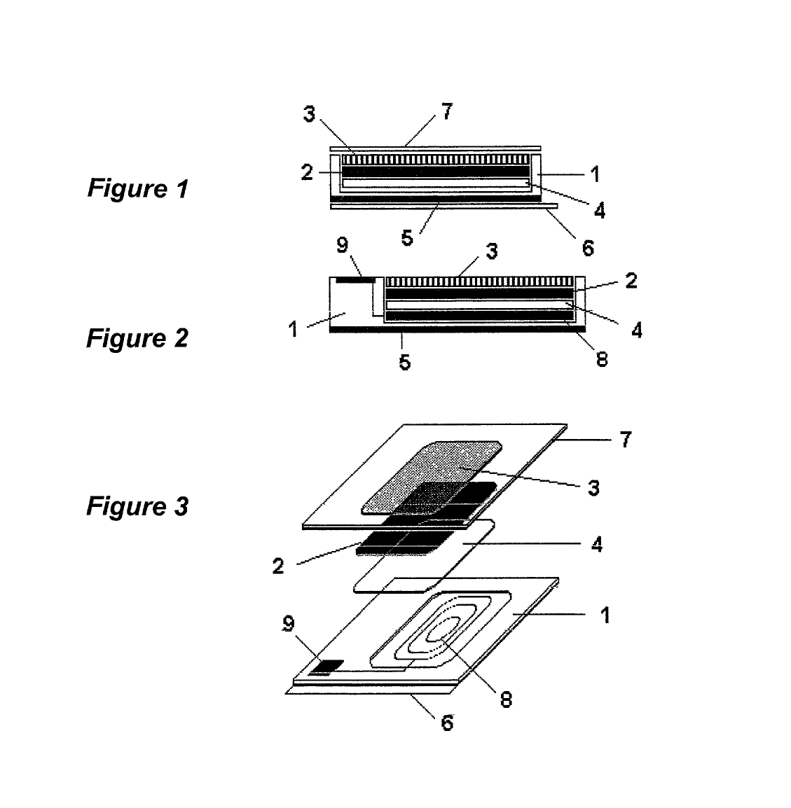

FIG. 1 shows, in schematic cross-section, the basic structure of a chip system. In a commonly shared chip carrier matrix (1), a carrier layer with chemosensorially active substances (2) is found. The latter is, in turn, embedded between an emission control layer (3), embodied as a membrane, which lies above it, and a promoter layer (4), which lies beneath it. The promoter layer (4) can contain, for example, solvents and/or a carrier vehicle for evaporation. The promoter layer (4) can be connected with reservoir layers for promoter substance, if necessary. On the underside of the chip carrier matrix, an adhesive layer (5) is found which, prior to use, is equipped with a covering layer (6). A removable covering layer (7) is found on the top of the emission control layer (3). After the removal of the covering layer, the emission of the chemosensory substances begins through the control membrane by diffusion. This process is supported and enhanced by promoter substances that are subsequently diffused into the substance layer.

In FIG. 2, the same chip system is supplemented by a thermo-active measure in which the promoter substance is heated in a controlled fashion by a heating coil (8). The thermo-active measure maybe any suitable heater. The guidance of control, energy supply, or other measurement procedures is undertaken by electronic microchips (9), which are integrated into the chip system.

FIG. 3 shows the same system, schematically once again, in an exploded schematic representation.

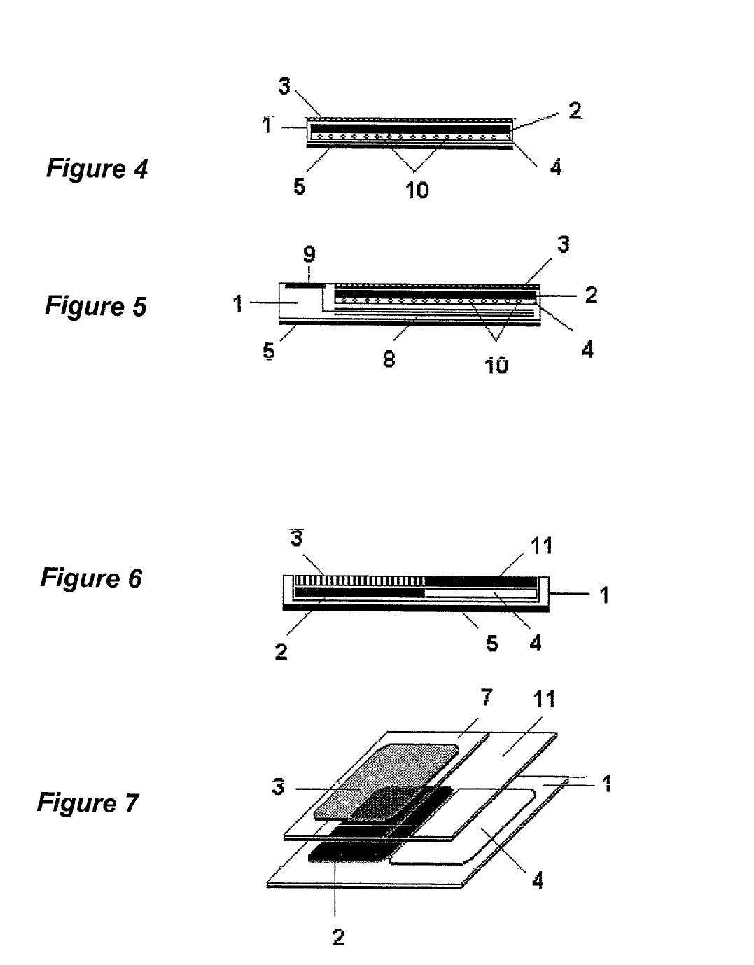

FIG. 4 shows a schematic structure in cross-section, in which the structure is realized by the use of a commonly shared matrix system. In this instance, the carrier matrix (1) is a polymer, which is shared commonly for all operational components, into which all components (2, 3, 4) are introduced, by strata, without a carrier substrate of its own. A reservoir function for the promoter substances (4) is realized in such a manner that the latter is present in two distinct phases. One phase is a rapidly diffusing form, the second phase releases on a delay, which can be realized, for example, in the form of delayed release carrier particles (10). The emission control layer corresponds to the material of the carrier polymer and is, on the top, applied only as a very thin layer. FIG. 5 shows the same schematic structure with supplement by means of an integrated active control unit (8, 9), as it is already depicted in FIG. 2.

FIG. 6 shows a parallel application of operational layers. The emission control component (3) and the chemosensory substance layer (2) are applied one atop the other, while the promoter layer (4) is applied in the same plane as the substance layer. Above the promoter layer, a covering (11) is found, which can be equipped as a thermal heat absorber. The underlying promoter layer is heated up, which leads to an elevated diffusion pressure toward the substance layer. This parallel embodiment form of the chemosensory chip systems can thus be created more flatly. FIG. 7 shows the same structure of FIG. 6 as an exploded schematic.

Obviously, numerous modifications and variations on the present invention are possible in light of the above teachings. It is therefore to be understood that within the scope of the appended claims, the invention may be practiced otherwise than as specifically described herein.

German patent application 100 61 057.9, filed Dec. 8, 2001, is incorporated herein by reference.

1. A chip system for the controlled emission of a substance having a chemosensory effect, comprising:
a chip carrier matrix;
a carrier layer comprising the substance having a chemosensory effect;
a promoter layer comprising an emission promoter; and
an emission control layer, which is a membrane or a polymer matrix.

2. The chip system according to claim 1, wherein the promoter layer is connected to an additional layer containing a promoter.

3. The chip system according to claim 1, further comprising an adhesive layer on an external underside of said chip carrier matrix; and
a covering layer which is removable from a top of said emission control layer.

4. The chip system according to claim 1, wherein said chip system further comprises a heater.

5. The chip system according to claim 1, wherein said promoter layer contains a delayed release carrier particle.

6. The chip system according to claim 1, further comprising an electronic microchip.

7. The chip system according to claim 1, wherein said promoter in said promoter layer is controlled mechanically, thermally, electrically, magnetically, biologically, chemically, bio-chemically, or by a combination of such measures;
wherein a regulation of a promotion of emission of said substance having a chemosensory effect occurs in a program series in an open loop or a closed loop technology.

8. The chip system according to claim 1, wherein said promoter is selected from the group consisting of an ethanol solution; a distilled oil; a synthetic chemical substance that possess a physicochemical property that is comparable to that of an ethanolic oil or a distilled oil; a technical gas; a natural gas; and a combination thereof;
wherein said promoter is initially present in the solid, liquid or gaseous state; and
wherein an individual promoter or a combination of promoters may be used.

9. The chip system according to claim 1, wherein said membrane or said polymer matrix consists of a natural substance or an artificial synthetic polymer;
wherein said membrane or said polymer matrix has at least one opening for the emission;
wherein said opening is applied by a mechanical aid or by a laser technique;
wherein a diameter of said opening is controlled dynamically and separately.

10. The chip system according to claim 1, wherein said carrier layer and said promoter layer are housed in a commonly shared matrix layer.

11. The chip system according to claim 1, wherein a spatial dimension of the chip system is in the range of 0.001 micrometer to 100 centimeter.

12. The chip system according to claim 1, wherein said chip system is directly combined with a technical device that influences or controls the quantity and composition of air.

13. The chip system according to claim 12, wherein said technical device influences one of a flow of the air, an air temperature, an air ionization, an air filtration, an aromatizing of the air, a humidity of the air or an admixture of air with a liquid, a solid, or a gas.

14. The chip system according to claim 1, wherein each component is made from a material selected from the group consisting of a flexible material, an expandable material, a transparent material, an electrically conductive material, a light-emitting material, a light absorbing material, a heat-storing material, a heat-emitting substance, a magnetic material, an electrically conductive metal foil, an electrically conductive plastic, an electronic component, a porous natural or synthetic polymer and a combination thereof.

15. The chip system according to claim 1, wherein said chip system is coupled with a device with which the content or the biological activity of a chemosensorially active substance or of its associated products or a product of its degradation is determined or displayed.

16. The chip system according to claim 1, which is coupled with at least one identical or different chip system to build a complex combination of chip systems.

17. The chip system according to claim 1, wherein said carrier layer comprises a natural or synthetic chemosensorially active substance that is suitable for influencing a disorder of the central or vegetative nervous system.

18. The chip system according to claim 17, wherein said disorder is a behavioral disorder, a symptom of stress, a sleep disorder, an anxiety disorder, an aggressive disorder, an eating disorder, an weight disorder, a sexual disorder, a reproductive disorder, pain, a vascular disorder, a circulatory disorder or a combination thereof.

19. The chip system according to claim 1, wherein said chemosensorially active substance is an active signaling substance in the human sphere, in the animal sphere, in the plant sphere, in an environmental system or in a biological regulatory process;
wherein said chemosensorially active substance or its natural or synthetic analog, its metabolite, its derivative, its isomer, its antagonist, or a combination thereof may be used.

20. The chip system according to claim 1, wherein said chemosensorially active substance, is contained one of a pharmaceutical product, a product of veterinary medicine, a medical aid, a product for health care, a product for care of the body, a cosmetic article, a perfumed article, a natural or artificial foodstuffs or beverage, a dietetic product, a plant, an agricultural product, a biological, microbiological, or chemical product for agricultural, horticultural, forest management and marine management, an agent to fend off or eliminate a harmful animal, a plant or a micro-organism, a household article, a sporting article, and an article for use, a packaging material, an article of clothing;
wherein said chemosensorially active substance or its natural or synthetic analog, its metabolite, its derivative, its isomer, its antagonist, or a combination thereof may be used.

21. A process for the controlled emission of a substance having a chemosensory effect, comprising:
releasing said substance or a mixture of at least two substances having a chemosensory effect from the chip system according to claim 1 as a volatile substance or a volatile mixture of at least two substances;
wherein a thermodynamic diffusion activity of said substance or said mixture of at least two substances is controlled by said promoter; and
wherein a rate of emission of said substance or said mixture of at least two substances is controlled by a layer that serves as a diffusion control;
wherein a promotion of said emission is governed chemically, physically, or biologically.

22. The process according to claim 21, wherein said promoter layer contains a delayed release carrier particle.

23. The process according to claim 21, wherein said promoter in said promoter layer is controlled mechanically, thermally, electrically, magnetically, biologically, chemically, bio-chemically, or by a combination of such measures;
wherein a regulation of a promotion of emission of said substance having a chemosensory effect occurs in a program series in an open loop or a closed loop technology.

24. The process according to claim 21, wherein said promoter is an ethanol solution; a distilled oil; a synthetic chemical substance that possess a physicochemical property that is comparable to an ethanolic oil or a distilled oil; a technical or natural gas;
wherein said promoter is initially present in the solid, liquid or gaseous state; and
wherein an individual promoter or a combination of promoters is used.

25. The process according to claim 21, wherein said chemosensorially active substance is suitable for influencing a disorder of the central or vegetative nervous system.

26. The process according to claim 25, wherein said disorder is a behavioral disorder, a symptom of stress, a sleep disorder, an anxiety disorder, an aggressive disorder, an eating disorder, an weight disorder, a sexual disorder, a reproductive disorder, pain, a vascular disorder, a circulatory disorder or a combination thereof.

27. The process according to claim 21, wherein said chemosensory active substance is an active signaling substance in the human sphere, in the animal sphere, in the plant sphere, in an environmental system or in a biological regulatory process;
wherein said chemosensorially active substance or its natural or synthetic analog, its metabolite, its derivative, its isomer, its antagonist, or a combination thereof is used.

28. The process according to claim 21, wherein said chemosensorially active substance, is contained in a pharmaceutical product, in a product of veterinary medicine, in a medical aid, in a product for health care or care of the body, in a cosmetic article or a perfumed article, in a natural or artificial foodstuffs or beverage, in a dietetic product, in a plant or an agricultural product, in a biological, microbiological, or chemical product for agricultural, horticultural, forest management and marine management, in a agent to fend off or eliminate a harmful animal, in a plant or a micro-organism, in a household article, in a sporting article, and in an article for use, in a packaging material, in an article of clothing;
wherein said chemosensorially active substance or its natural or synthetic analog, its metabolite, its derivative, its isomer, its antagonist, or a combination thereof is used.
